(19) United States
(12) Patent Application Publication
(10) Pub. No.: US 2018/0155757 A1
(43) Pub. Date: Jun. 7, 2018

(54) IDENTIFICATION OF BACTERIA THAT ARE RESISTANT TO CARBAPENEMS BY MEMBRANE IMPERVIOUSNESS

(71) Applicants: BIOMÉRIEUX, Marcy L'Etoile (FR); INSTITUT NATIONAL DE LA SANTE ET DE LA RECHERCHE MEDICALE (INSERM), Paris (FR); UNIVERSITE D'AIX MARSEILLE, Marseille (FR); SERVICE DE SANTÉ DES ARMÉES (SSA), Paris (FR)

(72) Inventors: Christine FRANCESCHI, Meximieux (FR); Elisabeth PINET, Angoulins (FR); Jean-Marie PAGES, Marseille (FR); Gilles ZAMBARDI, Tignieu (FR)

(73) Assignees: BIOMÉRIEUX, Marcy L'Etoile (FR); INSTITUT NATIONAL DE LA SANTE ET DE LA RECHERCHE MEDICALE (INSERM), Paris (FR); UNIVERSITE D'AIX MARSEILLE, Marseille (FR); SERVICE DE SANTÉ DES ARMÉES (SSA), Paris (FR)

(21) Appl. No.: 15/575,599

(22) PCT Filed: May 19, 2016
(86) PCT No.: PCT/FR2016/051180

§ 371 Date: Nov. 20, 2017

(30) Foreign Application Priority Data
May 20, 2015 (FR) 1554522

(51) Int. Cl. C12Q 1/18; C12Q 1/04; C12N 1/20

(52) U.S. Cl. CPC C12Q 1/18; A61K 31/397; C12N 1/20; C12Q 1/045

(57) ABSTRACT

A method for discriminating carbapenem-resistant bacteria according to whether or not they develop resistance by membrane impermeability, the method including the following steps: providing a bacterial strain that is resistant to at least one carbapenem, evaluating the sensitivity of the bacterial strain with regard to at least one indicator carbapenem, on the one hand in the absence of polymyxin B nonapeptide (PMBN) and, on the other hand, in the presence of PMBN in a non-inhibitory concentration, establishing a prognosis of resistance by membrane impermeability in the case where the presence of PMBN makes it possible to significantly increase the sensitivity of the bacterial strain with regard to at least one indicator carbapenem used.

The present invention belongs to the technical field of clinical microbiology, and relates to infectious disease screening and diagnosis techniques. It relates more particularly to the profiling of antibiotic-resistant bacteria and to the identification of the molecular mechanisms involved in this resistance.

Antibiotics have been used for medical purposes since the Second World War. Because of massive, repeated and sometimes indiscriminate use, both from a therapeutic point of view and from an industrial point of view (in particular for the preventive treatment of farm animals intended for the food chain), these molecules and the therapeutic efficacy thereof are today threatened with decline due to the emergence and expansion of bacterial strains that are increasingly resistant.

With a view to minimizing incorrect diagnoses and aimless treatments, manufacturers in the health field are working to assist practitioners in their function, by making available to them increasingly effective analytical methods and tools for detecting and identifying infectious microorganisms.

The present invention falls within the development of such analytical methods and tools and, more specifically, those which are dedicated to the profiling of bacteria that are termed antibiotic-resistant. More specifically, it relates to the methods and tools, the job of which is to characterize the type of molecular mechanism developed by certain bacteria in order to reduce their sensitivity to antibiotics.

There are many mechanisms for antibiotic resistance. Generally and schematically, they can be categorized according to the following resistance categories:
mutation or overexpression of the target of the antibiotic,
enzymatic inactivation of the antibiotic,
reduction of the cell-wall permeability
modification of the active efflux of antibiotics,
formation of a biofilm.

The prevalence of these mechanisms differs from one bacterium to another, but also from one type of antibiotic to another.

In the case of antibiotics of the carbapenem family (such as imipenem, meropenem, ertapenem, doripenem, tebipenem, faropenem), bacteria have mainly developed two types of resistance:
1) resistance by hydrolysis of the antibiotic, which is characterized by the production of enzymes with carbapenemase activity, and
2) resistance by reduction in membrane permeability; reference is then made to membrane impermeability.

The present invention focuses more particularly on the carbapenem sensitivity/resistance of bacteria. More specifically, it aims to determine whether or not a carbapenem-resistance developed by a bacterium can be attributed to a reduction in its membrane permeability. In other words, it aims to distinguish/discriminate, among bacteria which are carbapenem-resistant, those which are carbapenem-resistant by virtue of a mechanism of resistance by membrane impermeability.

Several analytical methods aimed at this same objective have already been described in the prior art. Most target porins, which are constituent membrane proteins of channels that antibiotics (and also other molecules) use to penetrate into bacteria. A faulty porin expression is thus used as an indicator of an antibiotic resistance by membrane impermeability.

For their implementation, these methods in particular call upon immunodetection techniques (ELISA assay, Western blot), molecular biology techniques (Southern blot, Northern blot, sequencing of nucleic sequences), mass spectrometry techniques, in particular MALDI-TOF mass spectrometry (Cai et al., Journal of Clinical Microbiology (2012), 50(6): 2179-82), for the purposes of physical detection of the porins, or of evaluation of the capacity of the bacteria tested to express these proteins.

These analytical methods, which target porins, have a major drawback: porins exist in many isoforms (in particular Omp1, Omp2, Omp35, Omp36, Omp37, OmpC, OmpD, OmpF, OmpN, OmpK35, OmpK36, OmpK37 etc.) the occurrence of which depends on and is a function of the bacterial genus and the bacterial species. Thus, unless antibodies, nucleic probes and/or reference MS spectra which enable the detection/identification of all of the existing porin isoforms are available, a reliable implementation of these methods requires not only a prior and precise identification of the bacterial genus to be analyzed, but also an exact knowledge of the particular isoforms expressed in this bacterial genus, and the availability of antibodies, nucleic probes and/or reference MS spectra that are suitable for the detection/identification of these particular isoforms.

Another drawback, which also affects the reliability of these methods, lies in the fact that a positive detection of porin expression does not make it possible to definitely dismiss a possible resistance by membrane impermeability. This is because it would, for this, also be necessary to verify that this porin expression is at a sufficient level and and to be sure that the proteins expressed are functionally assembled in the membrane.

The present invention aims to overcome the inconveniences, disadvantages and laboriousness that are encountered with the prior methods. In particular, it aims to provide a method for detecting and/or identifying bacteria which have developed carbapenem resistance by membrane impermeability, which can dispense with the great variability of the porin isoforms and with the need for verification of their expression at a functional and operational level.

Another objective of the present invention consists in providing an analytical method which can be carried out relatively simply and rapidly, and which does not require heavy, sophisticated and expensive technical equipment (such as for example mass spectroscopy equipment, etc.).

Another objective of the present invention consists in providing a method and associated tools which can be adapted and applicable to a large number of bacterial genera, species and strains.

The present invention thus provides a method for discriminating carbapenem-resistant bacteria according to whether or not they develop resistance by membrane impermeability, said method comprising the following steps:
providing a bacterial strain that is resistant to at least one carbapenem,
evaluating the sensitivity of said bacterial strain with regard to at least one indicator carbapenem, on the one hand in the absence of polymyxin B nonapeptide (PMBN) and, on the other hand, in the presence of PMBN in a non-inhibitory concentration;
establishing a prognosis of resistance by membrane impermeability in the case where the presence of PMBN makes it possible to significantly increase the sensitivity of said bacterial strain with regard to at least one indicator carbapenem used.

In the case where it is noted that the presence of PMBN does not make it possible to significantly increase the sensitivity of said bacterial strain to said indicator carbapenem(s), a prognosis of resistance other than membrane impermeability is then established. This is then very probably resistance by production of enzymes with carbapenemase activity.

In the context of the design of the present invention, it has been expressly demonstrated that a bacterium that is resistant to carbapenems by membrane impermeability experiences a significant increase in its carbapenem sensitivity, when it is treated with a particular permeabilizing agent, this being at particular concentrations of this permeabilizing agent. Conversely, this same permeabilization treatment, when it is applied to a bacterium developing another form of resistance, does not in any way produce such an effect.

The present invention thus provides a method for discriminating bacteria which have resistance by membrane impermeability, the principle of which consists in finding that it is actually possible to confer, on these bacteria, a level of permeability capable of re-establishing in them a “natural sensitivity” to carbapenems.

Thus, for each bacterium tested, the implementation of the discriminating method according to the invention makes it possible to obtain information relating to the sensitivity of the bacterium to an indicator carbapenem, which sensitivity is evaluated in the absence and in the presence of a non-inhibitory concentration of PMBN. Depending on this information, a prognosis is established with regard to the nature of the resistance mechanism developed by the bacterium.

In addition to the originality of the design which underlies the method provided, it is also to the inventors' credit to have identified, in PMBN, membrane permeabilization properties that are particularly suitable for the realization of this method and have thus diverted the use of this molecule, initially developed for exclusively therapeutic use, in order to make it an analytical tool.

Indeed, up until now, PMBN was known as being an antibiotic derived from polymyxin B. Like any polymyxin, it is a cyclic peptide with a hydrophobic tail, the antibiotic action of which results in denaturing destruction of the bacterial wall. The aminoacyl tail of PMBN is shorter than that of polymyxin B.

Like any polymyxin or other membrane-permeabilizing agent, its therapeutic use combined with other antibiotics has also been envisioned (WO 2008/017734). This being so, in addition to an accumulation of the therapeutic effects of each of the antibiotics used (including PMBN), a better penetration of antibiotics other than PMBN is also sought.

In the context of the present invention the inventors have been able to take advantage of the relatively “moderate” permeabilizing capacity of this molecule, this being a moderate permeabilizing capacity that makes it suitable for use at non-inhibitory concentrations in complex compositions, such as culture media. The amounts of PMBN required to obtain such concentrations in fact remain compatible with conventional techniques for mixing and forming complex compositions that are homogeneous in both chemical and biological terms.

In the context of the present invention, the significant nature of the increase in carbapenem sensitivity can be left to the operator to assess. Said operator, like any person skilled in the art, is able to distinguish an increase in sensitivity, the size of which is sufficiently large to attribute/ascribe it to an effect of the PMBN, rather than to “background noise” and/or to measurement or interpretation uncertainties that affect the conventional methods for evaluating antibiotic sensitivity of bacteria.

More objectively, for the purposes of the present invention, it is considered that the increase in sensitivity of a bacterial strain to an indicator carbapenem is significant as soon as a decrease in the corresponding (measured or evaluated) MIC of at least a factor of 2, or even of a factor of 3-4, is noted.

Before continuing with the description of the invention, the definitions hereinafter are given in order to facilitate the understanding and the disclosure of the invention.

The term “culture medium” is intended to mean a medium comprising the elements required for the survival and multiplication of microorganisms, in the case in point bacteria. The composition of a culture medium can comprise various constituents such as: amino acids, peptones, carbohydrates, nucleotides, minerals, vitamins, growth factors (sheep blood, horse blood), etc., and optionally dyes (for example, Evans blue, neutral red), opacifiers (for example, titanium oxide), metabolic indicators, metabolic regulators, etc.

A culture medium may be solid, semi-solid or liquid.

The term “solid culture medium” or “solid medium” is intended to mean for example a gelled medium. Agar is the conventional gelling agent in microbiology for culturing microorganisms, but it is also possible to use gelatin, agarose or other natural or artificial gelling agents. There are many commercially available culture media, for instance Columbia agar, Trypticase-soy agar, MacConkey agar, Mueller-Hinton agar or more generally those described in the Handbook of Microbiological Media (CRC Press).

The term “carbapenem-resistant bacterium” or more simply “resistant bacterium” is intended to mean a bacterium that has proven resistance for at least one carbapenem, regardless of the mechanism of resistance developed with regard to this antibiotic.

The term “non-inhibitory concentration of PMBN” is intended to mean a concentration of PMBN that produces, on a bacterium, a destructuring/denaturation of the membrane and which is not such that it lyses the bacterium or inhibits its multiplication. Objectively, for the purposes of the present invention, this “non-inhibitory” or “sub-inhibitory” concentration is more or less lower than, or equal to, the minimum inhibitory concentration (MIC) of PMBN for said bacterium.

The term “indicator carbapenem” is intended to mean an antibiotic belonging to the carbapenem class, which is used for carrying out the present invention and with regard to which the susceptibility/sensitivity of the bacterium to be tested is evaluated. The carbapenem chosen as indicator carbapenem, for the purposes of the present invention, does not necessarily correspond to the carbapenem or to one of the carbapenems to which the bacterium to be tested has developed a proven resistance. It is preferentially chosen for its broad spectrum of action, and its good chemical and enzymatic stability. Advantageously and according to the invention, each indicator carbapenem is preferentially chosen from: imipenem, ertapenem and meropenem.

According to one particular embodiment of the invention, the sensitivity of the bacterial strain to be tested is evaluated with regard to a single indicator carbapenem. A resistance by membrane impermeability is thus assigned to this bacterial strain provided that it is noted that the presence of PMBN makes it possible to significantly increase the sensitivity of this bacterial strain to this particular indicator carbapenem.

According to another embodiment of the invention, the sensitivity of the bacterial strain to be tested is evaluated with regard to several, preferably two, indicator carbapenems. In this case, a prognosis of resistance by membrane impermeability is established provided that it is noted that the presence of PMBN makes it possible to significantly increase the sensitivity of this bacterial strain with regard to at least one of the indicator carbapenems used.

As regards the non-inhibitory concentration of PMBN to be used for carrying out the present discriminating method, said concentration can be chosen specifically in relation to the bacterial strain to be tested, or non-specifically if the strain is not identified.

When it is specifically chosen, said non-inhibitory concentration of PMBN to be used results from the value of the MIC of PMBN determined/evaluated beforehand for the bacterial strain to be tested. Advantageously and according to the invention, said non-inhibitory concentration of PMBN is between about one tenth ( 1/10) and about half (½) of the MIC of PMBN for said bacterial strain. It is more preferentially chosen from one eighth (⅛) and one fifth (⅕) of said MIC.

The prior determination/evaluation of the MIC of PMBN can be carried out by any suitable method known to those skilled in the art, for example by:
a method of dilution (or of microdilution), in solid medium or in liquid medium,
a method of diffusion in which a disk of antibiotic or a strip of an antibiotic gradient, impregnated with PMBN, is used.

When the non-inhibitory concentration of PMBN to be used for carrying out the present invention is chosen non-specifically, in relation to the bacterial strain to be tested, it becomes needless to determine/evaluate beforehand the MIC of PMBN for said bacterial strain. Advantageously and according to the invention, a “standard” concentration of PMBN, advantageously of between 25 μg/ml and 500 μg/ml, preferentially of between about 150 μg/ml and about 350 μg/ml, will then be chosen. For most bacteria, in particular enterobacteria, such concentrations effectively correspond to non-inhibitory concentrations of PMBN for the purposes of the present invention.

As regards the evaluation of the sensitivity of the bacterial strain to each indicator carbapenem, this step can be carried out by any appropriate method known to those skilled in the art.

According to a first embodiment, this step is advantageously carried out by means of a method of diffusion on solid (culture) medium. To do this, each indicator carbapenem is used in the form of a disk of antibiotic or in the form of a strip of an antibiotic gradient, in particular Etest® strips (bioMérieux, France).

According to this first preferred embodiment, when the evaluation of the sensitivity of a bacterial strain to an indicator carbapenem is carried out in the presence of PMBN, the PMBN is initially present in said solid medium at a non-inhibitory concentration.

According to one implementation variant, the PMBN is not initially present in said solid medium, but is introduced therein extemporaneously and concomitantly with the indicator carbapenem(s). To do this, advantageously and according to the invention, disks of antibiotic or strips of antibiotic gradient, impregnated both with an indicator carbapenem and with PMBN, are used. Advantageously and according to one particular embodiment, as regards the PMBN, said disks and said strips have been preprepared by impregnation with a solution of PMBN having a PMBN concentration of between about 300 μg/ml and about 500 μg/ml.

According to a second preferred embodiment of the step of evaluating the sensitivity of a bacterial strain with regard to an indicator carbapenem, a method of microdilution in liquid medium is used. To do this, a standardized inoculant of the bacterial strain to be tested is brought into contact with a series of containers (such as, for example, tubes or cupules) containing a liquid culture medium with an increasing concentration of an indicator carbapenem. After incubation, the MIC is given by the container which contains the lowest concentration of antibiotic and in which no bacterial growth is visible. When this evaluation is carried out in the presence of PMBN, each container of culture medium also contains a non-inhibitory concentration of PMBN.

This non-inhibitory concentration of PMBN may be identical from one container to the other. According to one implementation variant, it advantageously varies from one container to the other and, for a given container, its value is indexed to the concentration of the indicator carbapenem of the culture medium contained in this container.

Advantageously and according to the invention, the evaluation of the sensitivity of the bacterial strain by the method of microdilution in liquid medium is carried out in an automated manner, for example by adapting automated devices, for example the VITEK® 2 AST (bioMérieux, France).

The present invention also relates to a kit which makes it possible to carry out a method for discriminating carbapenem-resistant bacteria according to whether or not they exhibit resistance by membrane impermeability.

According to a first embodiment, a kit according to the invention comprises at least:
one solid culture medium free of PMBN,
one solid culture medium comprising a non-inhibitory concentration of PMBN, and
two disks of antibiotic or two strips of antibiotic gradient, impregnated with an indicator carbapenem.

According to a second embodiment, a kit according to the invention comprises at least:
one solid culture medium free of PMBN,
two disks of antibiotic or two strips of antibiotic gradient, impregnated with an indicator carbapenem,
two disks of antibiotic or two strips of antibiotic gradient, impregnated both with an indicator carbapenem and with PMBN.

According to a third embodiment, a kit according to the invention comprises at least:
one first series of containers, in which each container contains a culture medium free of PMBN and comprising a concentration of an indicator carbapenem, the concentrations of indicator carbapenem of the various containers forming a concentration range;
one second series of containers, in which each container contains a culture medium, comprising an indicator carbapenem and a non-inhibitory concentration of PMBN, the concentrations of indicator carbapenem of the various containers forming a concentration range and the non-inhibitory concentrations of PMBN of the various containers being identical to one another or indexed to the concentration of indicator carbapenem of the culture medium contained in the container under consideration.

The invention also extends to a method for discriminating carbapenem-resistant bacteria according to whether or not they develop resistance by membrane impermeability, a method which makes it possible to establish a prognosis of resistance to carbapenems by membrane impermeability, and also a kit which makes it possible to carry out said discriminating and/or prognosis method, characterized by all or some of the technical features set out above and below.

Other objectives, features and advantages of the invention will emerge in the light of the description that follows and the examples, developed below, the objective of which is to facilitate the understanding of the invention and the implementation thereof. These examples are given by way of explanation and cannot limit the scope of the invention.

### EXAMPLES

1. Development of the Method According to the Invention on Bacterial Strains of Known Phenotype (“Porin+” or “Porine-”)

Firstly, carbapenem-resistant bacterial strains having a “porin+” and “porin−” phenotype were used as model strains for evaluating the relevance of the discriminating method according to the invention, and for characterizing the operating conditions suitable for the implementation thereof. This involved in particular testing various non-inhibitory concentrations of PMBN and various carbapenems as indicator carbapenems.

a) Model Strains Used

The bacterial strains used in this test come from collections belonging to the proprietors of the present industrial property title. These collections were put together over the years and bring together strains of microorganisms of various origins (clinical samples, international collections, and the like).

These resistant strains were pre-tested with respect to the presence or absence of porins in their membrane. To do this, Western blot analyses were carried out in order to detect the presence of the OmpF and OmpC isoforms, which are two isoforms known to be widely expressed by the bacterial genus to which these strains belong.

A strain is thus denoted “porin−” when neither of the two isoforms mentioned above could be detected. Resistance by membrane impermeability is then strongly suspected.

A strain is, on the other hand, denoted “porin+” when at least one of the two isoforms mentioned above could be detected. A mechanism of resistance other than membrane impermeability is then envisioned for this strain.

Likewise, prior to carrying out the discriminating method according to the invention, the PMBN-sensitivity of each of the model strains used was evaluated by means of a method of microdilution in liquid medium.

To do this, each of the strains was cultured in Mueller-Hinton broth (MHII-Becton Dickinson®, United States). On inocula containing 2×10⁵ CFU/ml, serial doubling dilutions were carried out for the PMBN in order to determine the minimum inhibitory concentrations. The PMBN-sensitivity was read after 18 hours of incubation at 37° C., by addition of 0.2 mg/ml of iodonitrotetrazolium chloride (Sigma-Aldrich®, United States).

Listed in table 1 below are the model strains used, their observed phenotype, and also their MIC with respect to PMBN.

TABLE 1

| Strains tested | Phenotype | MIC_{PMBN} (μg/ml) |
|---|---|---|
| Enterobacter aerogenes EA 2 | porin+ | 1024 |
| Enterobacter aerogenes EA 5 | porin− | 1024 |
| Enterobacter aerogenes EA 27 | porin− | 256 |
| Enterobacter aerogenes EA 15038 | porin+ | 256 |

b) Method Carried Out with Various Indicator Carbapenems According to the Invention and Various Non-Inhibitory Concentrations of PMBN

Firstly, two carbapenems were tested as indicator carbapenems according to the invention: imipenem (IM) and ertapenem (ETP).

For each model strain, the MIC with respect to each of these two carbapenems was determined in the absence and in the presence of PMBN (at non-inhibitory concentrations).

The determinations of antibiotic MIC were carried out on solid culture media with the Etest® method. To do this, Etest® strips (bioMérieux, France) were used:
Etest® Imipenem IM 32, and
Etest® Ertapenem ETP 32,
   and also solid culture media, differing from one another by virtue of their non-inhibitory concentrations of PMBN.

These solid culture media are prepared in Petri dishes (0=145 mm; V=50 ml) on a base of MH II Agar (Becton Dickinson®, United States), more or less supplemented with PMBN. The PMBN concentrations range from 0 up to concentrations equivalent to one fifth of the MIC of PMBN (⅕ MIC_{PMBN}).

Using a swab and the Retro C80™ rota-plater (bioMérieux, France), the culture media were inoculated with the bacteria, originating from an inoculum of 0.5 McFarland, prepared in 0.85% NaCl solution from colonies subcultured the day before on MH II agar.

The various Etest® strips are then deposited on the surface of the culture media. After 16 to 20 hours of incubation at 35° C., the MICS were read.

Table 2 below summarizes the results obtained (the MICs are expressed therein in μg/ml)

TABLE 2

| [PMBN] | 0 | MIC/10 | MIC/8 | MIC/5 | 0 | MIC/10 | MIC/8 | MIC/5 |
|---|---|---|---|---|---|---|---|---|
| | Enterobacter aerogenes EA 2 | | | | Enterobacter aerogenes EA 15038 | | | |
| | (porin+) | | | | (porin+) | | | |
| | MIC_{PMBN} = 1024 μg/ml | | | | MIC_{PMBN} = 256 μg/ml | | | |
| MIC_{IM} | 0.03 | 0.06 | 0.06 | 0.03 | 0.25 | 0.5 | 0.5 | 0.25 |
| MIC_{ETP} | 0.03 | 0.25 | 0.25 | 0.125 | 0.25 | 0.125 | 0.125 | 0.06 |
| | Enterobacter aerogenes EA 27 | | | | Enterobacter aerogenes EA 5 | | | |
| | (porin−) | | | | (porin−) | | | |
| | MIC_{PMBN} = 1024 μg/ml | | | | MIC_{PMBN} = 1024 μg/ml | | | |
| MIC_{IM} | 8 | 8 | 0.06 | 0.03 | 4 | 2 | 2 | 2 |
| MIC_{ETP} | 64 | 16 | 0.25 | 0.125 | 32 | 4 | 4 | 4 |

In the light of these first results, it appears that the carbapenem-resistant bacteria, exposed to a non-inhibitory concentration of PMBN, respond differently to imipenem (IM) and ertapenem (ETP), depending on the mechanism of resistance that they have developed with regard to carbapenems.

With the exception of the results obtained with the EA 15038 strain evaluated with ertapenem, it is thus noted that the strains having developed a resistance by membrane impermeability (“porin−” strains) experience a significant increase in their carbapenem sensitivity in the presence of a non-inhibitory concentration of PMBN (reflected by a decrease in the MIC_{IM} and MIC_{ETP}). Conversely, for the strains having developed another form of resistance, their MIC_{IM} and MIC_{ETP} remain unchanged or have a tendency to increase.

These first results confirm the potential that there is in using a combination of a non-inhibitory concentration of PMBN and an indicator carbapenem, for the purposes of the present invention, for establishing a prognosis for/identifying the mechanism of carbapenem resistance developed by a bacterium, in particular an enterobacterium.

2. Validation and Optimization of the Method According to the Invention

Secondly, the discriminating method according to the invention was carried out, “blind”, on various carbapenem-resistant bacterial strains (among these strains, some were identified beforehand as carbapenemase producers).

To do this, imipenem and ertapenem were used as indicator carbapenems. Non-inhibitory concentrations of PMBN, equivalent to concentrations of one tenth ( 1/10), one eighth (⅛) and one fifth (⅕) of the MIC of PMBN, were applied.

For each strain tested, each PMBN concentration and each indicator carbapenem, the MIC measured is compared to that obtained in the absence of PMBN. In the light of the possible difference in MIC values, a prognosis for the mechanism of resistance developed by the strain tested is established by applying the following general premise: A decrease in MIC_{IM/ETP} in the presence of PMBN is an indication of carbapenem resistance by membrane impermeability. In any other case, another mechanism of resistance is implicated.

For the purposes of verifying the accuracy of the prognoses established and the relevance of the general premise mentioned above:
each strain was tested by Western blot with the view to detection of porins (in the case in point, of the OmpF and OmpC isoforms), and
for some of the strains tested, in particular those for which there remains a doubt as to the mechanism of resistance developed (prognosis established in contradiction with the results of the porin detection by Western blot), a test for detecting carbapenemases by means of the RAPIDEC® CARBA NP kit (bioMérieux, France) was also carried out.

As regards the results thus obtained, the characterization of the strains termed carbapenem resistant by membrane impermeability is established either by the absence of porins detected by Western blot, or by carbapenem resistance that is not linked to the presence of carbapenemase, verified by a molecular method (PCR) and/or by a phenotypic method (RAPIDEC® CARBA NP).

The MIC_{ICM/ETP} measurements and the Western blot results thus obtained are compiled in table 3 below.

TABLE 3

| | E. coli 0506040 | | | | | | | | | |
|---|---|---|---|---|---|---|---|---|---|---|
| | (VIM-1 producer) | | | | | K. pneumoniae 1002015 | | | | |
| | MIC_{PMBN} = 128 μg/ml | | | | | MIC_{PMBN} = 1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 4 | 4 | 4 | 4 | Porin+ | 2 | 0.5 | 0.25 | 0.25 | Porin −; |
| MIC_{ETP} | 0.25 | 0.25 | 0.25 | 0.25 | | 32 | 4 | 2 | 1 | Carbapenemases not |
| | | | | | | | | | | detected |
| | S. marcescens 1008175 | | | | | K. pneumoniae 1012335 | | | | |
| | (IMP-1 producer) | | | | | (OXA-48 producer) | | | | |
| | MIC_{PMBN} = 1024 μg/ml | | | | | MIC_{PMBN} = 1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 128 | 128 | 256 | 256 | Porin+ | 2 | 2 | 2 | 2 | Porin+ |
| MIC_{ETP} | 256 | 256 | 256 | 256 | | 2 | 4 | 4 | 4 | |
| | K. pneumoniae 1104053 | | | | | K. pneumoniae 1108001 | | | | |
| | (OXA-48 producer) | | | | | (KPC-2 producer) | | | | |
| | MIC_{PMBN} = 1024 μg/ml | | | | | MIC_{PMBN} = 1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 2 | 2 | 2 | 2 | Porin+ | 8 | 8 | 8 | 4 | Porin+ |
| MIC_{ETP} | 4 | 4 | 4 | 4 | | 16 | 16 | 8 | 8 | |
| | | | | | | K. pneumoniae 1108016 | | | | |
| | E. aerogenes 1204048 | | | | | (VIM-1 producer) | | | | |
| | MIC_{PMBN} >1024 μg/ml | | | | | MIC_{PMBN} = 512 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 16 | 2 | 1 | 0.5 | Porin− | 4 | 8 | 8 | 4 | Porin+ |
| MIC_{ETP} | 32 | 4 | 2 | 2 | | 1 | 4 | 4 | 2 | |
| | E. coli 1204043 | | | | | K. pneumoniae 1204034 | | | | |
| | MIC_{PMBN} = 512 μg/ml | | | | | MIC_{PMBN} = 256 ng/ml | | | | |
| West.blot | 0 | MIC/10 | MIC/8 | MIC/5 | Verifications | 0 | MIC/10 | MIC/8 | MIC/5 | Verifications |
| MIC_{IM} | 8 | 2 | 1 | 1 | Porin− | 2 | 0.5 | 0.5 | 0.5 | Porin −; |
| MIC_{ETP} | 32 | 1 | 2 | 1 | | 64 | 8 | 4 | 4 | Carbapenemases not |
| | | | | | | | | | | detected |
| | K. pneumoniae 1108007 | | | | | | | | | |
| | (VIM-1 producer) | | | | | C. freundii 1204047 | | | | |
| | MIC_{PMBN} = 1024 μg/ml | | | | | MIC_{PMBN} = 1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 32 | 16 | 16 | 16 | Porin+ | 1 | 2 | 1 | 1 | Porin+−; |
| MIC_{ETP} | 8 | 16 | 16 | 8 | | 32 | 8 | 8 | 4 | Carbapenemases not |
| | | | | | | | | | | detected |
| | E. aerogenes 9203076 | | | | | E. cloacae 9304026 | | | | |
| | MIC_{PMBN} = 1024 μg/ml | | | | | MIC_{PMBN} >1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 8 | 2 | 2 | 2 | Porin− | 8 | 4 | 4 | 4 | Porin +; |
| MIC_{ETP} | 32 | 4 | 4 | 2 | | 16 | 4 | 8 | 8 | Carbapenemases not |
| | | | | | | | | | | detected |
| | E. aerogenes 9306071 | | | | | E. cloacae 9402034 | | | | |
| | MIC_{PMBN} = 1024 μg/ml | | | | | MIC_{PMBN} >1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 8 | 4 | 4 | 2 | Porin −; | 16 | 8 | 8 | 8 | Porin −; |
| MIC_{ETP} | 32 | 4 | 4 | 4 | Carbapenemases not | 64 | 16 | 16 | 16 | Carbapenemases not |
| | | | | | detected | | | | | detected |
| | K. pneumoniae 1108002 | | | | | | | | | |
| | (KPC-3 producer) | | | | | C. freundii 0403012 | | | | |
| | MIC_{PMBN} >1024 μg/ml | | | | | MIC_{PMBN} = 256 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 16 | 16 | 16 | 16 | Porin− | 4 | 2 | 1 | 1 | porin +; |
| MIC_{ETP} | 16 | 64 | 64 | 64 | | 8 | 8 | 4 | 4 | Carbapenemases not |
| | | | | | | | | | | detected |
| | K. pneumoniae 0404024 | | | | | E. cloacae 0901026 | | | | |
| | (VIM-1 producer) | | | | | (KPC-2 producer) | | | | |
| | MIC_{PMBN} >1024 μg/ml | | | | | MIC_{PMBN} = 1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 64 | 64 | 64 | 64 | Porin+ | 16 | 16 | 8 | 8 | Porin+ |
| MIC_{ETP} | 128 | 32 | 32 | 16 | | 16 | 32 | 32 | 8 | |
| | K. pneumoniae 0901027 | | | | | E. cloacae 1008073 | | | | |
| | (KPC-3 producer) | | | | | (VIM-1 producer) | | | | |
| | MIC_{PMBN} >1024 μg/ml | | | | | MIC_{PMBN} = 1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 8 | 16 | 8 | 16 | Porin+ | 0.25 | 0.25 | 0.25 | 0.25 | Porin+ |
| MIC_{ETP} | 16 | 64 | 64 | 64 | | 2 | 1 | 1 | 1 | |
| | E. coli 1008077 | | | | | E. cloacae 1008079 | | | | |
| | (VIM-1 producer) | | | | | (IMP-1 producer) | | | | |
| | MIC_{PMBN} = 1024 μg/ml | | | | | MIC_{PMBN} >1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 16 | 8 | 16 | 16 | Porin+ | 2 | 4 | 2 | 2 | Porin+ |
| MIC_{ETP} | 8 | 4 | 4 | 4 | | 4 | 8 | 8 | 8 | |
| | E. cloacae 1008096 | | | | | K. pneumoniae 1108006 | | | | |
| | (VIM-1 producer) | | | | | (KPC-2 producer) | | | | |
| | MIC_{PMBN} = 1024 μg/ml | | | | | MIC_{PMBN} >1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 4 | 8 | 8 | 4 | Porin+ | 32 | 32 | 64 | 32 | Porin+ |
| MIC_{ETP} | 4 | 4 | 4 | 4 | | 64 | 256 | 256 | 256 | |
| | E. cloacae 1101172 | | | | | E. coli 1012292 | | | | |
| | (NDM producer) | | | | | (KPC-2 producer) | | | | |
| | MIC_{PMBN} = 1024 μg/ml | | | | | MIC_{PMBN} = 1024 μg/ml | | | | |
| [PMBN] | 0 | MIC/10 | MIC/8 | MIC/5 | Verifications | 0 | MIC/10 | MIC/8 | MIC/5 | Verifications |
| MIC_{IM} | 4 | 8 | 16 | 4 | Porin+ | 8 | 4 | 4 | 1 | Porin+ |
| MIC_{ETP} | 4 | 8 | 8 | 4 | | 8 | 4 | 4 | 2 | |

a) Interpretation of the Results Obtained, Evaluation of the Prognoses Established

The notion of “reliability” used below, for quantifying the relevance of the discriminating method according to the invention and the accuracy of the interpretation of the results, refers to a correlation between the interpretation of the MIC measurements given by said method and the results of a physical detection of porins and of carbapenemases. It is not therefore an “absolute” reliability, but a relative reliability, a “pseudo-reliability”.

It should also be kept in mind that the presence of porins in the membrane of the strains screened provides only a presumption with regard to the absence of resistance by membrane impermeability, but does not provide uncontestable evidence (unless confirmation is also obtained of a sufficient presence of these porins, both in terms of number and in terms of activity). On the other hand, an absence of detection of carbapenemase allows a strong presumption of resistance by membrane impermeability.

Despite everything, the verification by a physical detection of porins and of carbapenemases, carried out here, provides a good standard of evaluation of the performance levels and of the relevance of the present method for discriminating mechanisms of carbapenem resistance developed by the bacteria. It is thus considered that the characterization of the strains termed carbapenem resistant by membrane impermeability is established either by the absence of porins detected by Western blot, or by carbapenem resistance that is not linked to the presence of carbapenemase, verified by a molecular method (PCR) and/or by a phenotypic method (RAPIDEC® CARBA NP).

In the light of the results presented in table 3, the following is noted:
Of the 168 MIC_{IM} and MIC_{ERT} values, measured in the presence of PMBN at non-inhibitory concentrations, 133 are in accordance with the general premise stated above. An interpretation of the results obtained by the discriminating method according to the invention, based on this general premise, shows a correlation of about 79% with the verifications carried out by Western blot, and optionally with the RAPIDEC® CARBA NP test. The correlation is approximately 81% with imipenem and approximately 77% with ertapenem.

In this regard, it should be noted that for the C. freundii 1204047, E. cloacae 9304026 and C. freundii 0403012 strains, given the absence of carbapenemase detected by the RAPIDEC®CARBA NP test, there is a strong presumption with regard to resistance by membrane impermeability. The porins detected by Western blot in fact appear to be qualitatively and/or quantitatively non-functional.
Of these 168 values, 87 do not correspond to a decrease in MIC_{IM/ETP} and imply a mechanism of resistance other than membrane impermeability, for the strains in question. The verifications carried out made it possible to confirm the accuracy of 77 of these prognoses. Thus, with this method, a negative prognosis with regard to resistance by membrane impermeability offers a reliability of about 89% (87% with imipenem and 90% with ertapenem).
Of these 168 values, 81 values correspond to a decrease in MIC_{IM/ETP} and imply resistance by membrane impermeability, for the strains in question. The verifications carried out made it possible to confirm the accuracy of 56 of these prognoses. With the method described, a positive prognosis with regard to resistance by membrane impermeability therefore exhibits a reliability of about 69% (73% with imipenem and 66% with ertapenem).

Thus, a discriminating method according to the invention, to which is applied a rule of interpretation of the indicator carbapenem MIC values noted, in accordance with the general premise stated above, should be able to offer a general reliability of about 79%. This reliability should be about 89% when a prognosis of resistance other than membrane impermeability is established. When a prognosis of membrane impermeability is established, it is then recommended to undertake additional confirmation tests.

B) Other Method of Interpretation of the Results Obtained, Evaluation of the Prognoses Established by Application of a Refined Premise

A second method of interpretation of the results compiled in table 3 is proposed here. The prognosis system is based on the following “refined” premise: A decrease in MIC_{IM/ETP}, in the presence of PMBN, by at least a factor of 4 (that is to say with a difference in MIC of 2 doubling dilutions), is the signal of carbapenem resistance by membrane impermeability. If not, another mechanism of resistance is implicated.

Taking into consideration this refined premise and in the light of the results presented in table 3, the following is noted:
Of the 168 MIC_{IM} and MIC_{ERT} values, measured in the presence of PMBN at non-inhibitory concentrations, 140 are in accordance with the refined premise stated above. An interpretation of the results obtained by the discriminating method according to the invention, based on this general premise, shows a correlation of about 83% with the verifications carried out by Western blot, and optionally with the RAPIDEC® CARBA NP test. The correlation is approximately 81% with imipenem and approximately 86% with ertapenem.
Of these 168 values, 120 do not correspond to a decrease in MIC_{IM/ETP} by at least a factor of 4 and imply a mechanism of resistance other than membrane impermeability, for the strains in question. The verifications carried out made it possible to confirm the accuracy of 92 of these prognoses. With this method, a negative prognosis with regard to resistance by membrane impermeability therefore exhibits a reliability of about 81% (77% with imipenem and 85% with ertapenem).
Of these 168 values, 48 correspond to a decrease in MIC_{IM/ETP} by at least a factor of 4 and imply a resistance other than by membrane impermeability, for the strains in question. The verifications carried out made it possible to confirm the accuracy of 43 of these prognoses. Thus, with this method, a positive prognosis with regard to resistance by membrane impermeability exhibits a reliability of about 90% (95% with imipenem and 86% with ertapenem).

Thus, a discriminating method according to the invention, to which is applied a rule of interpretation of the indicator carbapenem MIC values noted, in accordance with the refined premise stated above, should exhibit an improved overall reliability, that would now be about 83%.

This improvement is to the benefit of the prognoses that are positive for membrane impermeability, the reliability of which goes from 69% to 90%. It is apparently obtained to the detriment of the prognoses that are negative for membrane impermeability, the reliability of which, previously stated as 89%, decreases slightly and is now 81%.

c) Additional Observations

In the light of the two premises above, an intermediate interpretation rule would be capable of enabling further improvement in the reliability of the present method, namely:
a decrease in MIC_{IM/ETP}, in the presence of PMBN, by at least a factor of 4 (that is to say with a difference in MIC of 2 doubling dilutions), is the signal of carbapenem resistance by membrane impermeability,
an absence of decrease in MIC_{IM/ETP} in the presence of PMBN is the signal of a mechanism of resistance other than membrane impermeability,
a decrease in MIC_{IM/ETP} in the presence of PMBN, by a factor of less than 4, is the signal of a mechanism of resistance by membrane impermeability; it is nevertheless recommended to carry out additional tests.

Moreover, although lower with imipenem, the performance levels observed with imipenem and ertapenem, in the context of the implementation of the discriminating method according to the invention, are very similar and comparable. This suggests the possibility of using other carbapenems as indicator carbapenems according to the invention.

The above data demonstrate the relevance of the present method for discriminating the mechanism of carbapenem resistance developed by a bacterium. A certain modularity is permitted with regard to the choice of the indicator carbapenem to be used, and with regard to the non-inhibitory concentration of PMBN to be applied.

A certain modularity is also permitted with regard to the interpretation of the MIC variations observed, as a function of the indicator carbapenem and of the non-inhibitory concentration of PMBN applied, and optionally according to the characteristic to be demonstrated as a priority (resistance by membrane impermeability or other mechanism) and the level of reliability desired for this demonstration.

3. Simplification and Optimization of the Method According to the Invention

For the purpose of simplifying the implementation of this method, a generalization study was undertaken in order to identify one or more “standard” non-inhibitory concentration(s) of PMBN, that can advantageously be applied to a large number of bacteria. The determination of such standard concentrations would make it possible to avoid having to evaluate, for each bacterial strain to be tested, its level of sensitivity to PMBN.

In the light of the MIC_{PMBN} values previously identified, very predominantly much higher than 300 μg/ml, the following standard concentrations were retained and applied to the bacterial strains to be tested: 50, 100, 150, 200 and 300 μg/ml.

The validation of these standard concentrations of PMBN was carried out with bacterial strains, from collections belonging to the proprietors of the present industrial property title, and expressing a “porin+” or “porin−” membrane permeability phenotype.

By way of indicator carbapenems according to the invention, in addition to imipenem (IM) and ertapenem (ETP), meropenem (MP) was also tested. Like the other two antibiotics, meropenem was used in the form of Etest® strips (Etest® Meropenem MP 32 from the company bioMérieux, France).

The methods used to prepare the new culture media and to carry out the antibiotic-sensitivity tests are identical to those described above.

Table 4 below compiles the results obtained in a first series of measurements in which only “porin−” strains are tested (the MICS and the PMBN concentrations are expressed therein in μg/ml).

TABLE 4

| [PMBN] | 0 | 50 | 100 | 150 | 200 | 0 | 50 | 100 | 150 | 200 |
|---|---|---|---|---|---|---|---|---|---|---|
| | E. aerogenes EA 5 | | | | | E. aerogenes EA 27 | | | | |
| | (porin−) | | | | | (porin−) | | | | |
| MIC_{IM} | 6 | 4 | 4 | 4 | 3 | 12 | 6 | 3 | 3 | 3 |
| MIC_{ETP} | 6 | 2 | 2 | 1.5 | 1 | >32 | 4 | 1.5 | 0.75 | 0.19 |
| MIC_{MP} | 3 | 2 | 1.5 | 0.75 | 0.75 | 4 | 0.75 | 0.5 | 0.19 | 0.094 |
| | K. pneumoniae KP 55 | | | | | K. pneumoniae KP 63 | | | | |
| | (porin−) | | | | | (porin−) | | | | |
| MIC_{IM} | 0.38 | 0.25 | 0.25 | 0.25 | 0.19 | 0.25 | 0.25 | 0.125 | 0.125 | 0.125 |
| MIC_{ETP} | 0.38 | 0.25 | 0.19 | 0.125 | 0.064 | 1 | 0.50 | 0.38 | 0.19 | 0.125 |
| MIC_{MP} | 0.19 | 0.125 | 0.064 | 0.064 | 0.047 | 0.25 | 0.25 | 0.094 | 0.064 | 0.023 |
| | E. coli SAB 100 | | | | | E. coli SAB 108 | | | | |
| | (porin−) | | | | | (porin−) | | | | |
| MIC_{IM} | 0.5 | 0.38 | 0.25 | 0.19 | 0.19 | 0.75 | 0.38 | 0.25 | 0.25 | 0.19 |
| MIC_{ETP} | 1.5 | 0.19 | 0.19 | 0.125 | 0.125 | 4 | 0.25 | 0.19 | 0.19 | 0.19 |
| MIC_{MP} | 0.125 | 0.094 | 0.047 | 0.032 | 0.032 | 2 | 0.125 | 0.125 | 0.094 | 0.094 |
| | E. coli 1204043 | | | | | E. aerogenes 9203076 | | | | |
| | (porin−) | | | | | (porin−) | | | | |
| MIC_{IM} | 24 | — | — | 1 | 0.25 | 6 | — | — | 3 | 3 |
| MIC_{ETP} | >32 | — | — | 0.38 | 0.19 | 24 | — | — | 3 | 1.5 |
| MIC_{MP} | 3 | — | — | 0.064 | 0.008 | 4 | — | — | 0.5 | 0.5 |
| | K. pneumoniae 1002015 | | | | | K. pneumoniae 1204034 | | | | |
| | (porin−) | | | | | (porin−) | | | | |
| MIC_{IM} | 0.5 | — | — | 0.38 | 0.25 | 2 | — | — | 0.38 | 0.38 |
| MIC_{ETP} | 4 | — | — | 0.5 | 0.38 | >32 | — | — | 1.5 | 1 |
| MIC_{MP} | 2 | — | — | 0.19 | 0.19 | 12 | — | — | 0.5 | 0.25 |

Table 4a below compiles the results obtained in a second series of tests in which bacterial strains having proven membrane impermeability and carbapenemase-producing strains were tested.

TABLE 4a

| | K. pneumoniae 1204035 | | | | K. pneumoniae 1204036 | | | |
|---|---|---|---|---|---|---|---|---|
| | (membrane impermeability) | | | | (membrane impermeability) | | | |
| [PMBN] | 0 | 100 | 200 | 300 | 0 | 100 | 200 | 300 |
| MIC_{IM} | 1.5 | 0.75 | 0.5-0.75 | 0.5-1 | 2 | 2 | 2 | 1.5 |
| MIC_{ETP} | >32 | 8 | 8-12 | 6 | >32 | >32 | >32 | >32 |
| MIC_{MP} | 8-12 | 2 | 1.5 | 1.5 | 32 | 16 | 6 | 4 |
| | E. cloacae 1204051 | | | | E. cloacae 1204052 | | | |
| | (membrane impermeability) | | | | (membrane impermeability) | | | |
| [PMBN] | 0 | 100 | 200 | 300 | 0 | 100 | 200 | 300 |
| MIC_{IM} | 1.5 | 1.5 | 0.75 | 0.75 | 3 | 3 | 2 | 1.5 |
| MIC_{ETP} | >32 | 12 | 4 | 3 | >32 | 12 | 8 | 3 |
| MIC_{MP} | 4 | 2 | 0.25 | 0.25 | 6 | 0.75 | 1.5 | 0.5 |
| | E. cloacae 1204053 | | | | E. cloacae 1204056 | | | |
| | (membrane impermeability) | | | | (membrane impermeability) | | | |
| [PMBN] | 0 | 100 | 200 | 300 | 0 | 100 | 200 | 300 |
| MIC_{IM} | 3 | 3 | 3 | 3 | 6 | 2 | 1.5 | 1 |
| MIC_{ETP} | >32 | >32 | >32 | 4 | >32 | 12 | 3 | 1 |
| MIC_{MP} | 4 | 2 | 2 | 1.5 | 12 | 2 | 1 | 0.75 |
| | E. cloacae 1204058 | | | | K. pneumoniae 1204039 | | | |
| | (membrane impermeability) | | | | (membrane impermeability) | | | |
| [PMBN] | 0 | 100 | 200 | 300 | 0 | 100 | 200 | 300 |
| MIC_{IM} | >32 | 12 | 4 | 3 | 0.38 | 0.38 | 0.38 | 0.38 |
| MIC_{ETP} | >32 | 8-12 | — | 0.75 | 2 | 1.5 | 1.5 | 1 |
| MIC_{MP} | >32 | 2 | 0.75 | 0.25 | 0.75 | 0.5 | 0.38 | 0.38 |
| | E. aerogenes 1204050 | | | | E. cloacae 1204153 | | | |
| | (membrane impermeability) | | | | (membrane impermeability) | | | |
| [PMBN] | 0 | 100 | 200 | 300 | 0 | 100 | 200 | 300 |
| MIC_{IM} | >32 | >32 | >32 | >32 | 4 | 4 | 2 | 4 |
| MIC_{ETP} | >32 | >32 | >32 | >32 | >32 | 8 | 4 | 3 |
| MIC_{MP} | >32 | 2-4 | 1.5-2 | 1 | 4 | 0.75-1 | 1 | 0.75 |
| | E. cloacae 9402034 | | | | E. aerogenes 9306071 | | | |
| | (membrane impermeability) | | | | (membrane impermeability) | | | |
| [PMBN] | 0 | 100 | 200 | 300 | 0 | 100 | 200 | 300 |
| MIC_{IM} | >32 | 8 | 12 | 12 | 12 | 6 | 3 | 2 |
| MIC_{ETP} | >32 | >32 | 12 | 12 | >32 | 12 | 8 | 4 |
| MIC_{MP} | 16 | 2 | 2 | 3 | 4 | 1.5 | 1.5 | 0.5 |
| | E. aerogenes 9203076 | | | | K. pneumoniae 1204034 | | | |
| | (membrane impermeability) | | | | (membrane impermeability) | | | |
| [PMBN] | 0 | 100 | 200 | 300 | 0 | 100 | 200 | 300 |
| MIC_{IM} | 8 | 16 | 4 | 4 | 3 | 0.5 | 0.38 | 0.19 |
| MIC_{ETP} | >32 | 16 | 12 | 4 | >32 | 12 | 3 | 2 |
| MIC_{MP} | 4 | 2 | 1.5 | 0.5-0.75 | 16 | 1.5 | 0.75 | 0.38 |
| | E. coli 1204043 | | | | E. aerogenes 1204048 | | | |
| | (membrane impermeability) | | | | (membrane impermeability) | | | |
| [PMBN] | 0 | 100 | 200 | 300 | 0 | 100 | 200 | 300 |
| MIC_{IM} | >32 | 6-8 | 3-6 | 3 | >32 | 12 | 6 | 3 |
| MIC_{ETP} | >32 | 6 | 2-4 | 1.5 | >32 | 16 | 16 | 8 |
| MIC_{MP} | 4 | 0.75 | 0.38 | 0.19 | >32 | 1.5 | 1 | 0.5-1 |
| | K. pneumoniae 1012142 | | | | E. cloacae 1008080 | | | |
| | (KPC-2 producer) | | | | (IMP producer) | | | |
| [PMBN] | 0 | 100 | 200 | 300 | 0 | 100 | 200 | 300 |
| MIC_{IM} | >32 | >32 | >32 | >32 | 1 | 12 | 3 | 6 |
| MIC_{ETP} | 32 | >32 | >32 | >32 | 16 | 24 | >32 | 12-24 |
| MIC_{MP} | >32 | >32 | >32 | >32 | 2 | 8 | 6 | 4-6 |
| | E. coli 1008078 | | | | K. pneumoniae 1108003 | | | |
| | (VIM producer) | | | | (KPC-3 producer) | | | |
| [PMBN] | 0 | 100 | 200 | 300 | 0 | 100 | 200 | 300 |
| MIC_{IM} | 0.38 | 1.5 | 0.75 | 0.38 | 6 | 8 | 12 | 12 |
| MIC_{ETP} | 2 | 0.75 | 0.5 | 0.38 | 8 | 24 | >32 | >32 |
| MIC_{MP} | 0.5 | 0.5 | 0.25 | 0.25 | 6 | >32 | >32 | 4 |
| | K. pneumoniae 1103183 | | | | E. cloacae 1109137 | | | |
| | (NDM producer) | | | | (OXA-48 producer) | | | |
| [PMBN] | 0 | 100 | 200 | 300 | 0 | 100 | 200 | 300 |
| MIC_{IM} | 24 | >32 | >32 | 6 | 1.5 | 4 | 2 | 1.5 |
| MIC_{ETP} | >32 | >32 | 32 | 32 | 24 | — | 12 | >32 |
| MIC_{MP} | >32 | >32 | 24 | >32 | 3 | 3 | 1.5 | 2 |

E. cloacae 1008074
(VIM producer)

| [PMBN] | 0 | 100 | 200 | 300 |
|---|---|---|---|---|
| MIC_{IM} | 3 | 3 | 8 | 6 |
| MIC_{ETP} | 0.38 | 0.5 | 0.5 | 0.5 |
| MIC_{MP} | 0.38 | 1.5 | 1 | 1.5 |

It emerges from tables 4 and 4a that the results obtained show good uniformity between the variations in MIC that are obtained with the three carbapenems tested (close to 90% agreement between the variations). This good uniformity confirms that the implementation of the discriminating method according to the invention can be generalized to other carbapenems. However, imipenem (IM) exhibits slightly lower performance levels.

The most reliable results are obtained at the highest non-inhibitory concentrations of PMBN, and even more so with meropenem (MP) and ertapenem (ETP).

In particular, by applying the “refined” interpretation rule stated above, for non-inhibitory concentrations of PMBN from 150 to 300 μg/ml meropenem resulted in the obtaining of expected prognoses at 81.8%, and ertapenem 81.5%.

4. Methods for Evaluating Antibiotic Sensitivity Other than the Conventional Etest® Method, and Validation of Other Embodiments

In the examples above, the discriminating method according to the invention was implemented with a step of evaluating the sensitivity of the bacteria to the indicator carbapenem carried out by means of a method of diffusion of the antibiotic on a solid culture medium (with or without PMBN), and using strips impregnated with a gradient of an indicator carbapenem (Etest® method).

Other variants of implementation of this step of evaluating the sensitivity of the bacteria to the indicator carbapenem were also tested, namely:
a method of diffusion of the antibiotic on a solid culture medium without PMBN, and using strips impregnated both with a carbapenem gradient and with a standard amount of PMBN;
an automated step of microdilution in liquid medium, carried out by adapting the VITEK® 2 AST instrumentation and technology (bioMérieux, France).

a) Diffusion of the Indicator Carbapenem and of PMBN from One and the Same Etest® Strip.

In this particular embodiment of the invention, the PMBN is not initially present in the culture medium but, just like the indicator carbapenem, it is extemporaneously provided by gradual diffusion from an impregnated strip.

The strips provided are impregnated both with an indicator carbapenem and with PMBN. These strips were prepared from Etest® strips from the company bioMérieux (France):
Etest® Imipenem IM 32,
Etest® Ertapenem ETP 32, and
Etest® Meropenem MP 32,
   which are impregnated with a solution of PMBN.

Several impregnating solutions, having different concentrations of PMBN (100, 200 and 300 μg/ml) were tested.

These strips which accumulate PMBN and indicator carbapenem are then tested for their ability to be able to implement the discriminating method according to the invention. To do this, bacterial strains of known resistance phenotype:
membrane impermeability, or
carbapenemase production, were used as models.

Table 5 below summarizes the results obtained. These results are expressed therein as [number of strains that tested positive for resistance by membrane impermeability, over number of strains tested] this being as a function of the indicator carbapenem used and of the concentration of PMBN of the solution for impregnating the Etest® strips.

TABLE 5

| | | Number of positive strains/number of strains tested | | | | | | | | |
|---|---|---|---|---|---|---|---|---|---|---|
| | | Impregnating solution: [PMBN] (μg/ml) | | | | | | | | |
| | | 100 | 200 | 300 | 100 | 200 | 300 | 100 | 200 | 300 |
| Model | Membrane | 4/9 | 6/9 | 6/9 | 11/15 | 13/15 | 14/15 | 10/16 | 10/16 | 13/16 |
| strains | impermeability | | | | | | | | | |
| | Carbapenemase | 0/3 | 0/3 | 1/3 | 0/3 | 0/3 | 0/3 | 0/6 | 1/6 | 1/6 |
| | production | | | | | | | | | |

| | | |
|---|---|---|
| Imipenem | Meropenem | Ertapenem |

The results thus obtained make it possible to validate this particular embodiment of the discriminating method according to the invention, in which PMBN and indicator carbapenem are provided extemporaneously to the solid culture medium, by means of an Etest® strip.

b) Evaluation of the susceptibility to the indicator carbapenem by microdilution in liquid medium.

In this particular embodiment of the invention, the evaluation of the susceptibility of the strain tested to the indicator carbapenem is carried out by means of a microdilution method. The culture medium is therefore no longer a solid medium, but a set of liquid media, having different indicator carbapenem concentrations.

In the case in point, the inventors chose to adapt the VITEK® 2 AST instrumentation and technology (bioMérieux, France) to the present discriminating method. In doing so, the step of evaluating the sensitivity of the strains tested to the indicator carbapenem is advantageously automated.

To do this, the VITEK® 2 AST cards comprise a set of cupules filled with a liquid culture medium comprising a range of concentrations of an indicator carbapenem according to the invention, and a fixed non-inhibitory concentration of PMBN.

Table 6 below summarizes the results obtained. These results are expressed therein as [number of strains that tested positive for resistance by membrane impermeability, over number of strains tested] this being as a function of the indicator carbapenem and of the non-inhibitory concentration of PMBN that are used.

TABLE 6

| | | Number of positive | | | | | |
|---|---|---|---|---|---|---|---|
| | | strains/number of strains tested | | | | | |
| | | [PMBN] (μg/ml) | | | [PMBN] (μg/ml) | | |
| | | 100 | 200 | 300 | 100 | 200 | 300 |
| Model | Membrane | 11/15 | 13/15 | 14/15 | 3/7 | 4/7 | 4/7 |
| strains | impermeability | | | | | | |
| | Carbapenemase | 0/3 | 0/3 | 0/3 | 1/4 | 0/4 | 1/4 |
| | production | | | | | | |

| | |
|---|---|
| Meropenem | Imipenem |

These first results confirm that the step of evaluating the sensitivity to the indicator carbapenem can be carried out by microdilution in liquid medium, in particular by going back to the VITEK® 2 AST instrumentation and technology (bioMérieux, France).

1. A method for discriminating carbapenem-resistant bacteria according to whether or not they develop resistance by membrane impermeability, the method comprising the following steps:
providing a bacterial strain that is resistant to at least one carbapenem,
evaluating the sensitivity of the bacterial strain with regard to at least one indicator carbapenem, on the one hand in the absence of polymyxin B nonapeptide (PMBN) and, on the other hand, in the presence of PMBN in a non-inhibitory concentration,
establishing a prognosis of resistance by membrane impermeability in the case where the presence of PMBN makes it possible to significantly increase the sensitivity of the bacterial strain with regard to at least one indicator carbapenem used.

2. The method as claimed in claim 1, wherein each indicator carbapenem is chosen from: imipenem, ertapenem and meropenem.

3. The method as claimed in claim 1, wherein the non-inhibitory concentration of PMBN is between about one tenth ( 1/10) and about half (½) of the minimum inhibitory concentration (MIC) of PMBN for the bacterial strain.

4. The method as claimed in claim 1, wherein the non-inhibitory concentration of PMBN is between about 25 μg/ml and about 500 μg/ml.

5. The method as claimed in claim 1, wherein the increase in the sensitivity of the bacterial strain to an indicator carbapenem is significant provided that a decrease in MIC of at least a factor of 2 is noted.

6. The method as claimed in claim 1, wherein the evaluation of the sensitivity of the bacterial strain to each indicator carbapenem is carried out by means of a method of diffusion on solid medium.

7. The method as claimed in claim 6, wherein each indicator carbapenem is used in the form of a strip of antibiotic gradient.

8. The method as claimed in claim 6, wherein the PMBN is initially present in the solid medium at a non-inhibitory concentration.

9. The method as claimed in claim 6, wherein the PMBN is provided to the solid medium extemporaneously and concomitantly with the indicator carbapenem(s).

10. The method as claimed in claim 1, wherein the evaluation of the sensitivity of the bacterial strain with regard to each indicator carbapenem is carried out by means of a method of microdilution in liquid medium.

11. A kit which makes it possible to carry out a discriminating method as claimed in claim 1, comprising at least:
one solid culture medium free of PMBN,
one solid culture medium comprising a non-inhibitory concentration of PMBN, and
two disks of antibiotic or two strips of antibiotic gradient, impregnated with an indicator carbapenem.

12. A kit which makes it possible to carry out a discriminating method as claimed in claim 1, comprising at least:
one solid culture medium free of PMBN,
two disks of antibiotic or two strips of antibiotic gradient, impregnated with an indicator carbapenem,
two disks of antibiotic or two strips of antibiotic gradient, impregnated both with an indicator carbapenem and with PMBN.

13. A kit which makes it possible to carry out a discriminating method as claimed in claim 1, comprising at least:
one first series of containers, in which each container contains a culture medium free of PMBN and comprising a concentration of an indicator carbapenem, the concentrations of indicator carbapenem of the various containers forming a concentration range;
one second series of containers, in which each container contains a culture medium, comprising an indicator carbapenem and a non-inhibitory concentration of PMBN, the concentrations of indicator carbapenem of the various containers forming a concentration range and the non-inhibitory concentrations of PMBN of the various containers being identical to one another or indexed to the concentration of indicator carbapenem of the culture medium contained in the container under consideration.
